SEQUENCE LISTING (4 sequences)

SEQ ID NO: 1 - DNA, 757 bp, Homosapiens
  CDS (3)..(368)
ggaaccgagaggctgagactaacccagaaacatccaattctcaaactgaagctcgcactctcgcctccagcatgaaagtctctgccgcccttctgtgcctgctgctcatagcagccaccttcattccccaagggctcgctcagccagatgcaatcaatgccccagtcacctgctgttataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagacttgaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtacaaaaaaaaaaaaaa

SEQ ID NO: 2 - protein, 122 aa, Homosapiens
NREAETNPETSNSQTEARTLASSMKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 3 - protein, 76 aa, Homo sapiens
QPDAINAPVTCCYNFTNRKISVQRLASYRRITSSKCPKEAVIFKTIVAKEICADPKQKWVQDSMDHLDKQTQTPKT

SEQ ID NO: 4 - protein, 69 aa, Artificial Sequence
  misc_feature (1)..(69): MCP-1 truncation
QVTCCYNFTNRKISVQRLASYRRITSSKCPKEAVIFKTIVAKEICADPKQKWVQDSMDHLDKQTQTPKT

(19) United States
(12) Patent Application Publication
(10) Pub. No.: US 2003/0129214 A1
(43) Pub. Date: Jul. 10, 2003

(54) Methods of enhancing the biocompatibility of an implantable medical device

(75) Inventors: Paul Bornstein, Seattle, WA (US); Themis Kyriakides, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: University of Washington

(21) Appl. No.: 10/339,778

(22) Filed: Jan. 9, 2003

Related U.S. Application Data
(60) Provisional application No. 60/347,560, filed on Jan. 10, 2002.

(51) Int. Cl. A61K 48/00; A61M 31/00

(52) U.S. Cl. 424/423; 514/44; 604/500

(57) ABSTRACT

In one aspect, the invention provides methods for enhancing the biocompatibility of a medical device implanted within a portion of a living body. The methods comprise the step of contacting the portion of a living body that is in contact with an implanted medical device with an amount of a monocyte chemoattractant protein antagonist effective to inhibit chronic inflammation at the site of implantation or encapsulation of the device. In another aspect, the invention provides implantable medical devices comprising: (a) a device body; and (b) a surface layer attached to the device body, said surface layer comprising an amount of an antagonist of monocyte chemoattractant protein antagonist sufficient to reduce a foreign body response against the device, wherein the device is adapted to be implanted within a portion of a living body.

### CROSS-REFERENCE TO RELATED APPLICATION

The present application claims the benefit of U.S. provisional patent application serial No. 60/347,560, filed Jan. 10, 2002, under 35 U.S.C. § 119.

### GOVERNMENT RIGHTS

This invention was made with government support under grant number AR45418 awarded by the National Institutes of Health, and under grant number EEC-9529161 awarded by the National Science Foundation. The government has certain rights in the invention.

### FIELD OF THE INVENTION

The present invention relates to methods for enhancing the biocompatibility of implantable medical devices.

### BACKGROUND OF THE INVENTION

The implantation of a medical device into a living body elicits a foreign body response which results in the encapsulation of the implant by a poorly vascularized, collagenous capsule that can compromise the function of the device. In addition, the continued presence of the implanted device can lead to a chronic inflammatory response that is mediated, in part, by macrophages. The foreign body response has been implicated in the failure of numerous devices including glucose sensors, cochlear implants, breast augmentation prosthesis and artificial joints (Tang et al., Am J Clin. Pathol. 103:466-471 (1995); Woodward et al., Diabetes Care 5:278-281 (1982); Ratner, J. Biomed Mater Res. 27:837-850 (1993)). In the case of artificial joints, implant loosening is associated with the formation of foreign body giant cells and fibrotic capsules (Bostman et al. Clin. Orthop. 2000:216-227). Foreign body giant cells are multinucleated cells with normal ploidy which are derived from the fusion of activated macrophages recruited to the site of implantation as blood-borne monocytes. The presence of foreign body giant cells at the tissue-implant interface is a hallmark of the chronic inflammatory state that occurs in response to an implanted device (Anderson, Curr. Opin. Hematol. 7:40-47 (2000)).

Monocyte chemoattractant protein (MCP-1) is a chemoattractant cytokine that promotes the migration and activation of monocytes and has been associated with several inflammatory diseases such as rheumatoid arthritis (Koch et al., J. Clinical Invest. 90:772-779 (1992)). MCP-1 belongs to a small family of CC-type chemokines, (including MCP-1 through MCP-5) that can be synthesized by most cell types and interacts with the chemokine receptor CCR2, which is found primarily on monocytes and memory T lymphocytes. CC-type chemokine genes are found clustered at the chromosomal locus 17q11.2-12 and are called CC due to the presence of two adjacent cysteines at their amino-terminal end. Mature human MCP-1 is secreted as a 76 amino acid protein with two internal disulphide bridges between conserved cysteine residues (Cys34-Cys35 and Cys 35-Cys79). The amino terminal end of MCP-1 is responsible for the chemoattractant activity (Proost et al., J. Immun. 160:4034-41 (1998)). The present inventors have discovered that MCP-1 null mice exhibit minimal encapsulation and decreased chronic inflammation following implantation of a foreign body in comparison to wild-type control mice.

### SUMMARY OF THE INVENTION

In accordance with the foregoing, in one aspect the present invention provides methods of enhancing the biocompatibility of a medical device implanted within a portion of a living body. The methods comprise contacting a portion of a living body that is in contact with an implanted medical device with an amount of a monocyte chemoattractant protein (MCP-1) antagonist effective to inhibit a process selected from the group consisting of chronic inflammation induced by the presence of the medical device and fibrous encapsulation of the medical device, thereby enhancing the biocompatibility of the medical device. In some embodiments of this aspect of the invention, an MCP-1 antagonist and the medical device are separately introduced into the portion of the living body that contacts the implanted device (e.g., the device is implanted into the living body, and thereafter the MCP-1 antagonist is applied to the outside of the implanted device, thereby forming a layer between the implanted device and the surrounding tissue). In some embodiments the medical device further comprises an external surface layer comprising an MCP-1 antagonist. The methods of this aspect of the invention are applicable to enhance the biocompatibility of a medical device implanted into any living body, such as any animal, including mammals such as human beings.

In another aspect, the present invention provides implantable medical devices, each medical device comprising: (a) a device body; and (b) a surface layer attached to the device body, said surface layer comprising an amount of an antagonist of MCP-1 sufficient to reduce the foreign body response against the device, wherein the device is adapted to be implanted within a portion of a living body. Thus, in operation the surface layer contacts the portion of the living body into which the device is implanted, and reduces the foreign body response against the device.

The present invention also provides methods for making a biocompatible implantable medical device, the methods each including the step of making an implantable medical device including at least one external surface including a layer that includes at least one MCP-1 antagonist, to yield a biocompatible medical device.

The implantable medical devices of the invention and methods of making the devices are useful in any situation in which it is desirable to enhance the biocompatibility of an implanted medical device, such as in any living body, including human beings.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows a perspective view of a representative medical device of the invention with a portion of the surface layer removed to expose the underlying device body.

FIG. 2 shows a transverse cross-section of the medical device of FIG. 1.

FIG. 3 shows a porous matrix structure as a representative surface layer of the representative medical device shown in FIG. 1.

FIG. 4A shows a cross section of a filter after intraperitoneal implantation into a wild type mouse. The filter is encapsulated due to a foreign body reaction by the wild type mouse.

FIG. 4B shows a cross section of a filter after intraperitoneal implantation into an MCP-1-null mouse. The filter is substantially less encapsulated than the filter implanted into a wild type mouse (see FIG. 4A), due to a reduced foreign body reaction by the MCP-1-null mouse.

FIG. 5 shows a bar graph depicting reduced foreign body giant cell (FBGC) formation (per unit length of 200 μm as estimated by examination of hematoxylin and eosin stained tissue sections) following localized delivery of construct pMT-7ND expressing an amino-terminal truncated version of MCP-1 (MCP-1 lacking amino acids 2 through 8) as compared to delivery of control vector pCDNA3.

### DETAILED DESCRIPTION OF THE PREFERRED EMBODIMENT

Unless specifically defined herein, all terms used herein have the same meaning as they would to one skilled in the art of the present invention. The following definitions are provided in order to provide clarity with respect to the terms as they are used in the specification and claims to describe the present invention.

As used herein, the phrase “enhancing the biocompatibility of a medical device” refers to improving the acceptance of a medical device by a living body into which the device is physically implanted. A medical device with enhanced biocompatibility elicits a foreign body reaction that is less severe and/or of shorter duration than the foreign body reaction elicited by a control medical device that does not possess enhanced biocompatibility. By way of example, an implanted medical device with enhanced biocompatibility may elicit less severe chronic inflammation, and/or encapsulation of the device, in comparison to a control, implanted, medical device that does not possess enhanced biocompatibility.

As used herein, the term “foreign body response” refers to the biochemical and physiological repair processes characterized by chronic inflammation and/or encapsulation elicited at the site of implantation of a medical device.

The term “sequence identity” or “percent identical” as applied to nucleic acid molecules is the percentage of nucleic acid bases in a candidate nucleic acid molecule sequence that are identical with a subject nucleic acid molecule sequence (such as the nucleic acid molecule sequence set forth in SEQ ID NO: 1), after aligning the sequences to achieve the maximum percent identity, and not considering any nucleic acid base substitutions as part of the sequence identity. No gaps are introduced into the candidate nucleic acid sequence in order to achieve the best alignment.

Nucleic acid sequence identity can be determined, for example, in the following manner. The subject polynucleotide molecule sequence is used to search a nucleic acid sequence database, such as the Genbank database (accessible at Web site http://www.ncbi.nlm.nih.gov/blast/), using the program BLASTN version 2.1 (based on Altschul et al., Nucleic Acids Research 25:3389-3402 (1997)). The program is used in the ungapped mode. Default filtering is used to remove sequence homologies due to regions of low complexity as defined in Wootton, J. C. and S. Federhen, Methods in Enzymology 266:554-571 (1996). The default parameters of BLASTN are utilized. The BLASTN program compares the subject polynucleotide molecule sequence with polynucleotide molecule sequences stored in the database and provides a value for the percent identity between the compared sequences.

The term “sequence identity” or “percent identical” as applied to protein molecules is the percentage of amino acid residues in a candidate protein molecule sequence that are identical with a subject protein sequence (such as the protein sequence set forth in SEQ ID NO: 2), after aligning the sequences to achieve the maximum percent identity. No gaps are introduced into the candidate protein sequence in order to achieve the best alignment.

Amino acid sequence identity can be determined, for example, in the following manner. The subject protein sequence is used to search a protein sequence database, such as the GenBank database (accessible at web site http://www.ncbi.nln.nih.gov/blast/), using the BLASTP program. The program is used in the ungapped mode. Default filtering is used to remove sequence homologies due to regions of low complexity. The default parameters of BLASTP are utilized. Filtering for sequences of low complexity utilize the SEG program. The BLASTP program compares the subject protein sequence with protein sequences stored in the database and provides a value for the percent identity between the compared sequences.

The term “hybridize under stringent conditions,” and grammatical equivalents thereof, refers to the ability of a nucleic acid molecule to hybridize to a target nucleic acid molecule (such as a target nucleic acid molecule immobilized on a DNA or RNA blot, such as a Southern blot or Northern blot) under defined conditions of temperature and salt concentration. With respect to nucleic acid molecules greater than about 100 bases in length, typical stringent hybridization conditions are no more than 25° C. to 30° C. (for example, 10° C.) below the melting temperature (Tm) of the native duplex (see generally, Sambrook et al., Molecular Cloning: A Laboratory Manual, 2nd Ed. Cold Spring Harbor Press, 1987; Ausubel et al., Current Protocols in Molecular Biology, Greene Publishing, 1987). Tm for nucleic acid molecules greater than about 100 bases can be calculated by the formula Tm=81.5+0.41% (G+C−log (Na⁺)), wherein G is guanine and C is cytosine.

Exemplary hybridization and wash conditions include: hybridization at 65° C. in 5.0×SSC, 0.5% sodium dodecyl sulfate, for 16 hours, followed by two washes of thirty minutes each at 55° C. in 1.0×SSC, 1% (w/v) sodium dodecyl sulfate. Other exemplary hybridization and wash conditions include: hybridization at 65° C. in 5.0×SSC, 0.5% sodium dodecyl sulfate, for 16 hours, followed by two washes of thirty minutes each at 55° C. in 0.5×SSC, 1% (w/v) sodium dodecyl sulfate. Further exemplary hybridization and wash conditions include: hybridization at 65° C. in 5.0×SSC, 0.5% sodium dodecyl sulfate, for 16 hours, followed by two washes of thirty minutes each at 55° C. in 0.1×SSC, 1% (w/v) sodium dodecyl sulfate.

The abbreviation “SSC” refers to a buffer used in nucleic acid hybridization solutions. One liter of the 20× (twenty times concentrate) stock SSC buffer solution (pH 7.0) contains 175.3 g sodium chloride and 88.2 g sodium citrate.

The term “complement” when used in connection with a nucleic acid molecule refers to the complementary nucleic acid sequence as determined by Watson-Crick base pairing. For example, the complement of the nucleic acid sequence 5′CCATG3′ is 5′CATGG3′.

The term “antibody” encompasses polyclonal and monoclonal antibody preparations, CDR-grafted antibody preparations, as well as preparations including hybrid antibodies, altered antibodies, F(AB)′₂ fragments, F(AB) molecules, Fv fragments, single domain antibodies, chimeric antibodies and functional fragments thereof which exhibit immunological binding properties of the parent antibody molecule. The antibodies can also be humanized.

In one aspect, the present invention provides methods of enhancing the biocompatibility of a medical device implanted within a living body. The methods of this aspect of the invention comprise contacting the portion of the living body that contacts the implanted device with an amount of a monocyte chemoattractant protein (MCP-1) antagonist effective to inhibit chronic inflammation induced by the presence of the medical device, and/or fibrous encapsulation of the medical device. In the practice of the invention, contacting the portion of the body that contacts the implanted medical device with an MCP-1 antagonist can be achieved by any useful means. For example, in some embodiments of this aspect of the invention, an MCP-1 antagonist and a medical device are separately introduced into a portion of a living body. For example, a liquid preparation containing an MCP-1 antagonist can be injected, or otherwise introduced, into tissue surrounding an implanted medical device prior to, simultaneously with, or subsequent to implantation of a medical device into a living body. In some embodiments the medical device comprises at least one surface layer comprising an MCP-1 antagonist. The surface layer(s) contacts the portion of the body that contacts the medical device upon implantation. The methods of this aspect of the invention are applicable to any living body, such as any animal, including mammals, such as human beings.

The methods of this aspect of the invention can be used, for example, to enhance the biocompatibility of a medical device in any situation in which it is desirable to reduce chronic inflammation and/or encapsulation at the site of the implant, thereby prolonging the working lifetime and efficiency of the implanted device. The methods of the invention can be used to enhance biocompatibility of devices that are completely implanted into a living body (i.e., the entire device is implanted within a living body). Representative examples of completely implantable medical devices include, but are not limited to: cardiovascular devices (such as vascular grafts and stents), artificial blood vessels, prosthetic devices (such as artificial hip joints and artificial knee joints), and scaffolds that support tissue growth (in such anatomical structures as bone, tooth, nerves, pancreas, eye and muscle).

The methods of this aspect of the invention are also useful for enhancing biocompatibility of devices that are partially implanted within a living body (i.e., only part of the device is implanted within a living body, the remainder of the device being located outside of the body). Representative examples of partially implantable medical devices include, but are not limited to: biosensors (such as those used to monitor the level of drugs within a living body, or the level of blood glucose in a diabetic patient), percutaneous devices (such as catheters) that penetrate the skin and link a living body to a medical device such as a kidney dialysis machine, and skin substitutes (such as dermal and epidermal scaffolds).

Typically, the immediate biochemical result of implanting biomaterials in vivo is the deposition of proteins from plasma and tissue to the biomaterial surface. Shortly after implantation, monocytes are recruited to the site and differentiate into macrophages. Unable to digest the foreign material, the macrophages then fuse into foreign body giant cells and remain in juxtaposition to the implanted material surface indefinitely. As a result, the healing of the tissue surrounding the material is suspended and eventually the implant is walled off by development of a fibrotic capsule. The presence of foreign body giant cells at the site of an implant is therefore a hallmark for the chronic inflammatory response typically induced by an implanted medical device (Anderson, Curr. Opin. Hematol. 7:40-47 (2000)). In addition, foreign body giant cells have been implicated in degradation of biomaterial surfaces of implanted devices due to their high concentration of enzymes (Zhao et al., J. Biomed Mater Res. 25:177-183 (1991). Finally, foreign body giant cells are thought to be a source of chemokines, such as IL-8 which recruits neutrophils and lymphocytes to the site and may lead to deposition of collagen by fibroblasts leading to encapsulation (Anderson, supra).

The present inventors have discovered that implantation of foreign material in MCP-1 knockout mice results in a significant reduction of foreign body giant cells at the site of implantation in comparison to wild-type mice. The present inventors have also discovered that inhibition of MCP-1 activity at the surface of implantation results in less degradation of the surface of the implant, consistent with enhanced biocompatibilty. While not wishing to be bound by theory, the inventors hypothesize, based on these observations, that MCP-1 plays an important role in the process of macrophage fusion which is known to lead to the formation of foreign body giant cells. Therefore, the inventors have employed approaches that aim to inhibit MCP-1 expression or activity in tissues surrounding the implanted device in order to enhance the biocompatibility of the implanted device by reducing chronic inflammation and/or inhibiting the formation of fibrous encapsulation.

In accordance with the practice of the invention, the portion of a living body that contacts the implanted device is contacted with an amount of an MCP-1 antagonist effective to inhibit chronic inflammation and/or encapsulation of the device. In some embodiments of this aspect of the invention, the process of chronic inflammation typically induced by an implanted device is inhibited. In some embodiments fibrous encapsulation of the device is inhibited. In some embodiments, both chronic inflammation and fibrous encapsulation are inhibited.

An inhibition in chronic inflammation at the site of implantation can be identified, for example, by at least one of the following changes: a decrease in persistence of inflammatory cells (such as foreign body giant cells and/or activated macrophages) present at the site of implantation; a decrease in the level of cytokines, such as interleukin and monocyte chemoattractant protein in extracts of tissue taken from the site of implantation by ELISA; a decrease in the growth factors secreted by inflammatory cells, such as TGF-β; or a decrease in the amounts of proteolytic enzymes such as matrix metalloproteinases, collagenases, elastases and acid hydrolases (measured, for example, by analyzing tissue extracts by zymography, Western blot, ELISA, or immunohistochemical staining of tissue sections). Tissue extracts can also be analyzed by the PAI-1 lucifierase assay using mink lung epithelial cells (Kyriakides et al., Am. J. Pathol. 159:1255-1262 (2001)).

An inhibition of encapsulation at the site of implantation can be characterized, for example, by at least one of the following: a decrease in the amount of fibrosis (measured, for example, by determining the level of hydroxyproline content which indicates the level of collagen in the foreign body capsule); a decrease in capsule thickness (measured, for example, by examination of histological sections with the aid of an ocular micrometer); a decrease in the amount of contraction of collagen fibers within the capsule (measured, for example, as tensile strength of the capsule or induced shape change on malleable implants); or a decrease in the diffusion rates of small molecules through the capsule (measured, for example, as described by Sharkawy et al., J. Biomed. Mater. Res. 37:401-412 (1997)).

An MCP-1 antagonist useful in the practice of the invention is any molecule that inhibits MCP-1 protein expression or MCP-1 protein activity in a living body. In some embodiments of the methods of the present invention, an MCP-1 antagonist inhibits MCP-1 protein expression in the portion of a living body which contacts the implanted medical device. In the practice of the invention, representative MCP-1 antagonists useful for inhibiting MCP-1 protein expression include: antisense MCP-1 nucleic acid molecules (such as antisense mRNA, antisense DNA or antisense oligonucleotides), MCP-1 ribozymes, and molecules that cause RNA interference (RNAi) mediated inactivation of MCP-1 thereby preventing MCP-1 from eliciting a biological response. The methods of these embodiments of the invention can be used to enhance the biocompatibility of an implanted medical device by inhibiting the process of chronic inflammation and/or inhibiting encapsulation of the implanted device.

An antisense nucleic acid molecule may be constructed in any way such that it is capable of interfering with the expression of a target gene. For example, an antisense nucleic acid molecule can be constructed by inverting the coding region (or a portion thereof) of MCP-1 relative to its normal orientation for transcription to allow the transcription of its complement.

The antisense nucleic acid molecule is usually substantially identical to at least a portion of the target gene or genes. The nucleic acid molecule, however, need not be perfectly identical to inhibit expression. Generally, higher homology can be used to compensate for the use of a shorter antisense nucleic acid molecule. The minimal percent identity is typically greater than about 70%, but a higher percent identity may exert a more effective repression of expression of the endogenous sequence. Substantially greater percent identity of more than about 80% typically is preferred, though about 90% to about 95% to absolute identity is typically most preferred.

The antisense nucleic acid molecule need not have the same intron or exon pattern as the target gene, and non-coding segments of the target gene may be equally effective in achieving antisense suppression of target gene expression as coding segments. A DNA sequence of at least about 50 nucleotides may be used as the antisense nucleic acid molecule, although a longer sequence is preferable. In the present invention, a representative example of a useful antagonist of MCP-1 is an antisense MCP-1 nucleic acid molecule which is at least 70% identical (e.g., 80%, 90%, 95% or 99% identical) to the complement of the MCP-1 cDNA consisting of the nucleic acid sequence set forth in SEQ ID NO: 1 [from nucleotide 1-757]. The nucleic acid sequence set forth in SEQ ID NO: 1 encodes the human MCP-1 protein consisting of the amino acid sequence set forth in SEQ ID NO: 2. The amino acid sequence of the mature secreted form of human MCP-1 is set forth in SEQ ID NO: 3 (Furutani et al., Biochem. Biophys. Res. Commun. 159(1):249-255 (1989).

Useful antagonists include, for example, isolated nucleic acid molecules at least 100 bases in length that hybridize under stringent conditions to SEQ ID NO: 1. Useful antagonists also include nucleic acid molecules between 10 base pairs and 100 base pairs that hybridize at 10° C. below their melting temperature (Tm) to the complement of SEQ ID NO: 1. Other useful oligonucleotides include nucleic acid molecules between 10 bp and 100 bp that are at least 90% identical to any 10 bp portion of SEQ ID NO: 1 (such as to any 10 bp portion of SEQ ID NO: 1 extending from nucleotide 1-757). Tm for nucleic acid molecules greater than about 100 bases can be calculated by the formula Tm=81.5+0.41% (G+C−log (Na⁺)), wherein G is guanine and C is cytosine. With respect to nucleic acid molecules having a length less than 100 bases, exemplary stringent hybridization conditions are 5° C. to 10° C. below Tm. On average, the Tm of a nucleic acid molecule of length less than 100 bp is reduced by approximately (500/oligonucleotide length) degrees centigrade. Other useful antagonists are antisense MCP-1 nucleic acid molecules that include at least one portion that is at least 70% (such as at least 80%, at least 90%, at least 95% or at least 99%) identical to the complement of nucleic acid sequence of SEQ ID NO: 1.

The targeting of antisense oligonucleotides to bind MCP-1 mRNA is another mechanism that may be used to reduce the level of MCP-1 protein synthesis. For example, the synthesis of polygalacturonase and the muscarine type 2 acetylcholine receptor are inhibited by antisense oligonucleotides directed to their respective mRNA sequences (U.S. Pat. No. 5,739,119 and U.S. Pat. No. 5,759,829). Furthermore, examples of antisense inhibition have been demonstrated with the nuclear protein cyclin, the multiple drug resistance gene (MDG1), ICAM-1, E-selectin, STK-1, striatal GABA_{A} receptor and human EGF (see, e.g., U.S. Pat. No. 5,801,154; U.S. Pat. No. 5,789,573; U.S. Pat. No. 5,718,709 and U.S. Pat. No. 5,610,288).

Ribozymes can also be utilized to decrease the expression of MCP-1, such as ribozymes which target MCP-1 mRNA. Ribozymes are catalytic RNA molecules that can cleave nucleic acid molecules having a sequence that is completely or partially homologous to the sequence of the ribozyme. It is possible to design ribozyme transgenes that encode RNA ribozymes that specifically pair with a target RNA and cleave the phosphodiester backbone at a specific location, thereby functionally inactivating the target RNA. In carrying out this cleavage, the ribozyme is not itself altered, and is thus capable of recycling and cleaving other molecules. The inclusion of ribozyme sequences within antisense RNAs confers RNA-cleaving activity upon them, thereby increasing the activity of the antisense constructs.

Ribozymes useful in the practice of the invention typically comprise a hybridizing region, of at least about nine nucleotides, which is complementary in nucleotide sequence to at least part of the target MCP-1 mRNA, and a catalytic region which is adapted to cleave the target MCP-1 mRNA (see generally, EPA No. 0 321 201; WO88/04300; Haseloff & Gerlach, Nature 334:585-591 (1988); Fedor & Uhlenbeck, Proc. Natl. Acad. Sci.: USA 87:1668-1672 (1990); Cech & Bass, Ann. Rev. Biochem. 55:599-629 (1986)). Specific ribozyme cleavage sites within any potential RNA target can be identified by scanning the target RNA for ribozyme cleavage sites which include, for example, the following sequences: GUA, GUU, and GUC. Once identified, short RNA sequences of between 9 and 20 ribonucleotides corresponding to the region of the target polynucleotide containing the cleavage site can be evaluated for secondary structural features which can render the oligonucleotide inoperable. Antisense molecules and ribozymes of the invention can be prepared by any method known in the art for the synthesis of nucleic acid molecules.

RNA-mediated interference (RNAi), a form of gene silencing triggered by double-stranded (dsRNA) is also useful in the practice of the invention. The presence of dsRNA activates a host surveillance mechanism that targets mature, dsRNA-complementary mRNA for destruction. RNAi has been shown to work in mammalian cells provided that short interfering dsRNAs of about 21 nucleotides long are used that are specific to the gene of interest (see generally, Carthew, Curr Opin Biol. 13(2):244-8 (2001); Donze and Picard, Nucleic Acids Research 30:10 (2002)). Accordingly, dsRNA molecules useful for silencing MCP-1 expression in the practice of the method of the invention include dsRNA molecules of about 20-25 nucleotides, and more preferably 21 nucleotides which are complementary in nucleotide sequence to at least a portion of a target MCP-1 mRNA such as a target MCP-1 mRNA having the sequence set forth as SEQ ID NO: 1.

Methods of producing dsRNA include in vitro synthesis and in vivo transcription. For example, in vitro synthesis of dsRNA may be achieved by synthesizing sense and antisense RNA from DNA templates using T7 polymerase, followed by digestion of the DNA template with Dnase I and subsequent hybridization to form dsRNA. (See generally, Bhattacharyya et al., Nature 343:484 (1990)). In vivo transcription can be achieved by engineering a eukaryotic expression vector which contains promoters on opposite ends of a designated DNA sequence, such as a portion of the MCP-1 sequence, in which the promoters are oriented towards each other and capable of transcribing a strand of DNA into RNA. Consequently, the two resulting transcripts hybridize immediately between themselves in vivo, giving rise to a dsRNA molecule (See for example, U.S. Pat. No. 5,795,715).

In another embodiment of this aspect of the present invention, an MCP-1 antagonist inhibits MCP-1 activity in the portion of a living body which contacts the implanted medical device. In the practice of the invention, representative MCP-1 antagonists useful for inhibiting MCP-1 activity include, for example, an anti-MCP-1 antibody and an MCP-1 blocking peptide.

In one embodiment of the invention, the MCP-1 antagonist is an anti-MCP-1 antibody. By way of representative example, antigen useful for raising antibodies can be prepared in the following manner. A nucleic acid molecule (such as a cDNA molecule encoding MCP-1 set forth as SEQ ID NO: 1) is cloned into a plasmid vector, such as a Bluescript plasmid (available from Stratagene, Inc., La Jolla, Calif.). The recombinant vector is then introduced into an E. coli strain (such as E. coli XL1-Blue, also available from Stratagene, Inc.) and the polypeptide encoded by the nucleic acid molecule is expressed in E. coli and then purified.

For example, E. coli XL1-Blue harboring a Bluescript vector including a cDNA molecule of interest is grown overnight at 37° C. in LB medium containing 100 μg ampicillin/ml. A 50 μl aliquot of the overnight culture is used to inoculate 5 ml of fresh LB medium containing ampicillin, and the culture grown at 37° C. with vigorous agitation to A₆₀₀=0.5 before induction with 1 mM IPTG. After an additional two hours of growth, the suspension is centrifuged (1000×g, 15 min, 4° C.), the media removed, and the pelleted cells resuspended in 1 ml of cold buffer that preferably contains 1 mM EDTA and one or more proteinase inhibitors. The cells can be disrupted by sonication with a microprobe. The chilled sonicate is cleared by centrifugation and the expressed, recombinant polypeptide purified from the supernatant by art-recognized protein purification techniques. Alternatively, polypeptide fragments of MCP-1 can be prepared using peptide synthesis methods that are well known in the art. The synthetic polypeptides can then be used to prepare antibodies. Direct peptide synthesis using solid-phase techniques (Stewart et al., Solid-Phase Peptide Synthesis, W H Freeman Co, San Francisco Calif. (1969); Merrifield, J. Am. Chem. Soc. 85:2149-2154 (1963) is an alternative to recombinant or chimeric peptide production. Automated synthesis may be achieved, for example, using Applied Biosystems 431A Peptide Synthesizer (Foster City, Calif.) in accordance with the instructions provided by the manufacturer. Additionally the polypeptide sequences of the present invention or any fragment thereof may be mutated during direct synthesis and, if desired, combined using chemical methods with other amino acid sequences. The polypeptides used to induce specific antibodies may have an amino acid sequence consisting of at least five amino acids and preferably at least 10 amino acids. Short stretches of amino acid sequence may be attached with those of another polypeptide, and the chimeric polypeptide used for antibody production. Alternatively, the polypeptide may be of sufficient length to contain an entire domain for antibody recognition.

Methods for preparing monoclonal and polyclonal antibodies are well known to those of ordinary skill in the art and are set forth, for example, in chapters five and six of Antibodies A Laboratory Manual, E. Harlow and D. Lane, Cold Spring Harbor Laboratory (1988). Antibody production includes not only the stimulation of an immune response by injection into animals, but also analogous processes such as the production of synthetic antibodies, the screening of recombinant immunoglobulin libraries for specific-binding molecules (Orlandi et al., Proc. Natl. Acad. Sci. USA 86:3833, 1989, or Huse et al. Science 256:1275, 1989), or the in vitro stimulation of lymphocyte populations.

In another embodiment of the invention, MCP-1 activity is inhibited with MCP-1 blocking peptides that bind specifically to and inhibit, the active site of MCP-1, or an MCP-1 binding partner, or a receptor of MCP-1, such as CCR2. Human MCP-1 is secreted as a 76 amino acid protein, set forth as SEQ ID NO: 3. Chemical synthesis of MCP-1 analogues has revealed that the amino-terminal residues 1-6 are important for receptor recognition and signaling, and modification or removal of the amino terminal region can completely inactive these chemokines (Proost et al., J. Immun. 160:4034-41, (1998)). Examples of amino-terminal truncated versions of MCP-1 useful in the practice of this invention include the following MCP-1 blocking peptides (amino acid numbers are with reference to SEQ ID NO: 1): (MCP-1 residues 7-76), (MCP-1 residues 8-76), (MCP-1 residues 9-76), and an MCP-1 truncation lacking amino acid residues 2-8 (and including residues 1 and 9-76). In one embodiment of this aspect of the invention, the MCP-1 antagonist is MCP-1 lacking amino acids 2-8, set forth as SEQ ID NO: 4.

Other examples of blocking peptides useful in the practice of this invention include any peptides that block the activity of MCP-1, including for example, amino terminal deletions of MCP-2. Studies have shown that amino-terminal truncations of MCP-2, such as, for example, an MCP-2 truncation (including amino acid residues 6-76 of MCP-2) can completely block the chemotactic effect of MCP-1 on monocytes (Proost, supra). Other examples of useful peptide antagonists include MCP-1 fusion peptides, amino terminal modifications of MCP-1 such as N-terminal methylation, amino acid substitutions, glycosylation, proteolytic cleavage, and linkage to an antibody molecule or other cellular ligand.

The MCP-1 blocking peptides of the invention can be produced by chemical synthesis in accordance with art recognized methods and also by incorporating a nucleic acid molecule, encoding such as, for example, SEQ ID NO: 4, into an expression vector, introducing the expression vector into a host cell and expressing the nucleic acid molecule to yield polypeptide. The polypeptide can then be recovered and purified by any applicable purification method, including ammonium sulfate or ethanol precipitation, acid extraction, anion or cation exchange chromatography, gel filtration, hydrophobic interaction chromatography, affinity chromatography, hydroxyapatite chromatography, and high performance liquid chromatography (“HPLC”).

The MCP-1 blocking peptides can also be produced in vivo, for example by delivering a vector containing a DNA molecule encoding such as, for example, SEQ ID NO: 4 operationally linked to an expression cassette to the portion of the body in contact with the medical device, in accordance with the methods of the invention described herein.

In some embodiments of the practice of the invention, an MCP-1 antagonist is introduced into a living body separately from the implanted medical device. Introduction of the MCP-1 antagonist into the living body may occur prior to, simultaneous with, or subsequent to implantation of the medical device. Thus, for example, an MCP-1 antagonist may be formulated in a gelatinous composition that is first applied to a portion of a living body before implantation therein of a medical device. Again by way of example, a medical device may be implanted into a portion of a living body, and thereafter the gelatinous composition, including the MCP-1 antagonist, may be applied to the portion of the living body that is in contact with the implanted device. Any delivery method may be used to deliver MCP-1 antagonists to a living body, including methods for delivery of DNA encoding an MCP-1 antagonist, and methods for delivery of MCP-1 polypeptide antagonists such as MCP-1 blocking peptides.

Any art-recognized gene delivery method can be used to introduce a vector containing an MCP-1 antagonist into one or more cells for expression therein, including: transduction, transfection, transformation, direct injection, parenteral administration, electroporation, virus-mediated gene delivery, amino acid-mediated gene delivery, biolistic gene delivery, lipofection and heat shock (See, generally, Sambrook et al., supra). Examples of useful expression vectors include vectors derived from retrovirus, adenovirus (Ad), adeno-associated virus (AAV), herpes or vaccinia viruses, or from bacterial plasmids which can be used for delivery of nucleic acid molecules to cells in vivo.

Representative, non-viral methods of gene delivery into cells in vivo are disclosed in Huang, L., M.-C. Hung, and E. Wagner, Non-Viral Vectors for Gene Therapy, Academic Press, San Diego, Calif. (1999). For example, a vector may be formulated for delivery either encapsulated in a lipid particle, a liposome, a vesicle, or a gene activated collagen matrix. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Recently, liposomes were developed with improved serum stability and circulation half-times (see, e.g., U.S. Pat. No. 5,741,516). Furthermore, various methods of liposome and liposome-like preparations as potential drug carriers have been reviewed (see, e.g., U.S. Pat. Nos. 5,567,434; 5,552,157; 5,565,213; 5,738,868 and 5,795,587).

Various devices have been developed for enhancing the availability of DNA to a target cell. A simple approach is to contact the target cell physically with catheters or implantable materials containing DNA (G. D. Chapman et al., Circulation Res. 71:27-33 (1992)). Another method for achieving gene transfer involves using a fibrous collagen implant material soaked in a solution of DNA shortly before being placed in the site in which one desires to achieve gene transfer. The matrix may become impregnated with a gene DNA segment simply by soaking the matrix in a solution containing the DNA, such as a plasmid solution.

Expression vectors useful for expressing MCP-1 antagonists, or inhibitory fragments thereof, include chromosomal, episomal, and virus-derived vectors, e.g., vectors derived from bacterial plasmids, bacteriophages, yeast episomes, yeast chromosomal elements, viruses such as baculoviruses, papova viruses, vaccinia viruses, adenoviruses, fowl pox viruses, pseudorabies viruses and retroviruses, and vectors derived from combinations thereof, such as cosmids and phagemids. In certain embodiments in this regard, the vectors provide for specific expression, which may be inducible and/or cell type-specific. As referred to herein, the term “vector” refers to a nucleic acid molecule, usually double-stranded DNA, which may have inserted into it another nucleic acid molecule (the insert nucleic acid molecule) such as, but not limited to, a cDNA molecule. The vector is used to transport the insert nucleic acid molecule into a suitable host cell. A vector may contain the necessary elements that permit transcribing and translating the insert nucleic acid molecule into a polypeptide. The insert nucleic acid molecule may be derived from the host cell, or may be derived from a different cell or organism. Once in the host cell, the vector can replicate independently of, or coincidental with, the host chromosomal DNA, and several copies of the vector and its inserted nucleic acid molecule may be generated. Many molecules of the polypeptide (if any) encoded by the insert nucleic acid molecule can thus be rapidly synthesized.

Adenoviral vectors are designed to be administered directly to patients. Unlike retroviral vectors, adenoviral vectors do not integrate into the chromosome of the host cell. Instead, genes introduced into cells using adenoviral vectors are maintained in the nucleus as an extrachromosomal element (episome) that persists for a limited time period. Adenoviral vectors will infect dividing and non-dividing cells in many different tissues in vivo including airway epithelial cells, endothelial cells, hepatocytes and various tumors (B. C. Trapnell, Adv Drug Del Rev. 12:185-199 (1993)).

Another viral vector is the herpes simplex virus; a large, double-stranded DNA virus. Recombinant forms of the vaccinia virus can accommodate large inserts and are generated by homologous recombination. To date, this vector has been used to deliver, for example, interleukins (ILs), such as human IL-1β and the costimulatory molecules B7-1 and B7-2 (G. R. Peplinski et al., Ann. Surg. Oncol. 2:151-9 (1995); J. W. Hodge et al., Cancer Res. 54:5552-55 (1994)).

A plasmid vector can be introduced into mammalian cells in a precipitate, such as a calcium phosphate precipitate, or in a complex with a charged lipid (e.g., LIPOFECTAMINE™; Life Technologies, Inc.; Rockville, Md.) or in a complex with a virus (such as an adenovirus) or components of a virus (such as viral capsid peptides). If the vector is a virus, it may be packaged in vitro using an appropriate packaging cell line and then transduced into host cells.

MCP-1 polypeptide antagonists can be delivered into the portion of the body contacting the medical device by any suitable means such as delivery of polypeptide into the body in association with a pharmaceutical composition. By way of representative example, MCP-1 polypeptide antagonists can be introduced into a living body by direct application of a pharmaceutical composition comprising said MCP-1 antagonist to the portion of the living body that contacts the medical device. The polypeptides may be admixed or compounded with any conventional, pharmaceutically acceptable carrier or excipient. Compositions suitable for parenteral administration typically comprise sterile aqueous preparations of the agents which are preferably isotonic with the blood of the recipient. Suitable carrier solutions include, for example, phosphate buffered saline, saline, water, lactated ringers or dextrose (5% in water). Such compositions may optionally contain one or more additional ingredients among which may be for example, preservatives, such as methyl benzoate, chlorocresol, metacresol, phenol and benzalkonium chloride. Buffers may also be included to provide a suitable pH for the formulation. Suitable such materials include sodium phosphate and acetate. Sodium chloride or glycerin may be used to render the formulation isotonic with the blood.

MCP-1 polypeptide antagonists may be introduced in association with another molecule, such as a lipid, to protect the protein from enzymatic degradation. For example, the covalent attachment of polymers, especially polyethylene glycol (PEG), has been used to protect certain proteins from enzymatic hydrolysis in the body and thus prolong half-life (F. Fuertges, et al., J. Controlled Release, 11:139 (1990)). Many polymer systems have been reported for protein delivery (Y. H. Bae, et al., J. Controlled Release, 9:271 (1989); R. Hori, et al., Pharm. Res., 6:813 (1989); I. Yamakawa, et al., J. Pharm. Sci., 79:505 (1990); I. Yoshihiro, et al., J. Controlled Release, 10:195 (1989); M. Asano, et al., J. Controlled Release, 9:111 (1989); J. Rosenblatt et al., J. Controlled Release, 9:195 (1989); K. Makino, J. Controlled Release, 12:235 (1990); Y. Takakura et al., J. Pharm. Sci., 78:117 (1989); Y. Takakura et al., J. Pharm. Sci., 78:219 (1989)).

Methods of delivery of MCP-1 antagonist proteins, or fragments thereof, also include administration by oral, pulmonary, parenteral (e.g, intramuscular, intraperitoneal, intravenous (IV) or subcutaneous injection), inhalation (such as via a fine powder formulation), transdermal, nasal, vaginal, rectal, or sublingual routes of administration, and can be formulated in dosage forms appropriate for each route of administration. As used herein, the term “parenteral administration” of MCP-1 antagonist proteins and nucleic acids encoding MCP-1 antagonists includes any route of administration characterized by physical breaching of a tissue of a living body and administration of the MCP-1 antagonist by direct injection, by application through a surgical incision, by application through a tissue-penetrating non-surgical wound, by application using a device (e.g. a balloon angiocatheter) inserted at one site in a blood vessel of a living body and physically urged along a vessel to a second site), by administration of the MCP-1 antagonist using a wound dressing (e.g., a bandage, a suture, or a hernia repair mesh), and the like. In particular, parenteral administration is contemplated to include subcutaneous, intraperitoneal, intravenous, intraarterial, intramuscular or intrasternal injection techniques.

In some embodiments, the implantable medical device comprises a surface layer comprising an MCP-1 antagonist. The surface layer can be made from any suitable material such as, for example, a porous matrix, a hydrogel, or deposition of an MCP-1 antagonist onto a modified surface of a medical device. The devices disclosed and described herein are useful in the practice of this aspect of the invention.

In another aspect, the present invention provides biocompatible implantable medical devices comprising (a) a device body; and (b) a surface layer attached to the device body, said surface layer comprising an amount of an antagonist of MCP-1 sufficient to reduce the foreign body response against the device, wherein the device is adapted to be implanted within a portion of a living body (e.g., a mammalian body, such as a human body). Some medical devices of the invention are adapted to be completely implanted into a portion of a living body (i.e., the entire device is implanted within the body). Some medical devices of the invention are adapted to be partially implanted into a living body (i.e., only part of the device is implanted within a body with the remainder of the device being located outside of the body).

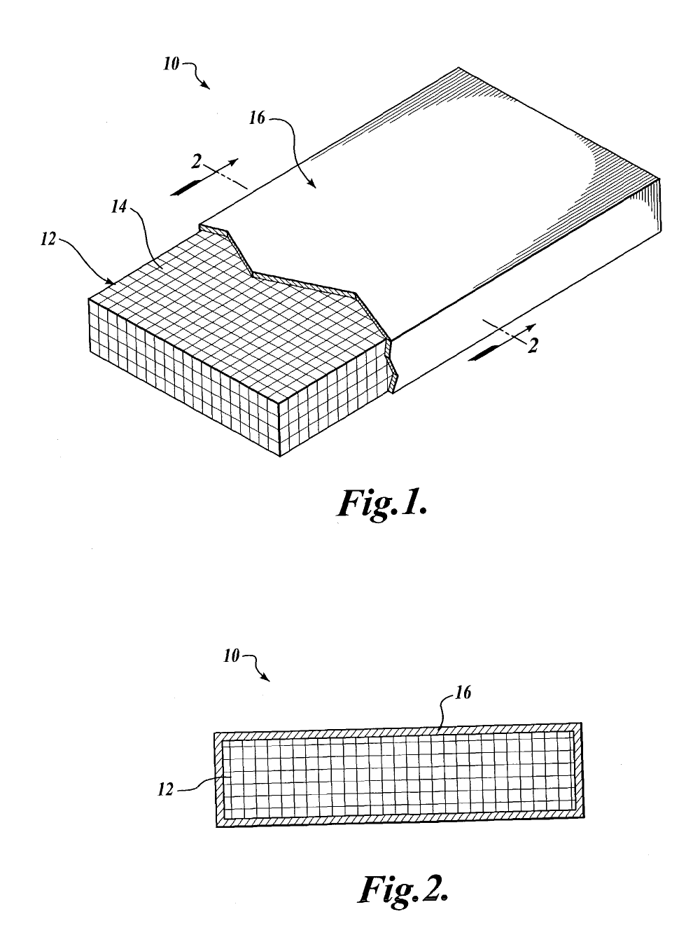

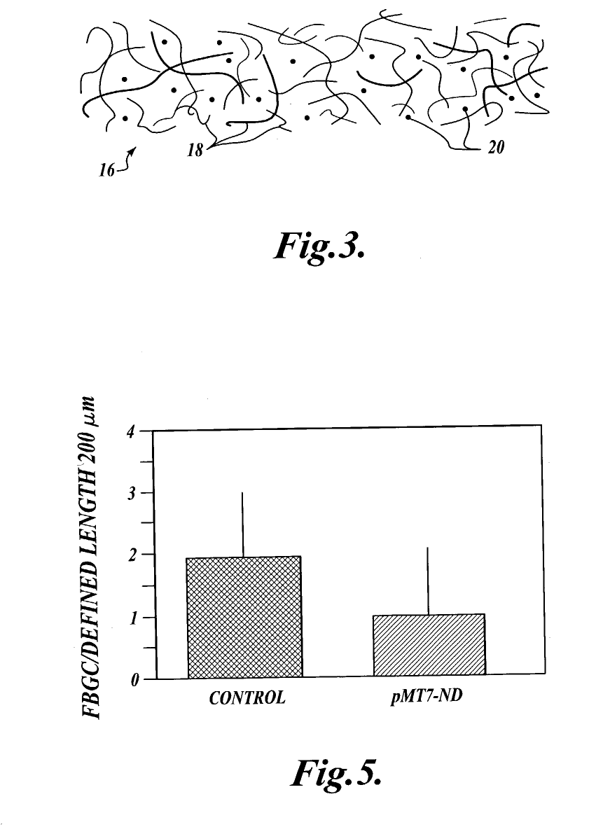

FIG. 1 shows a representative implantable medical device 10 of the present invention in the form of an implantable drug delivery device. Device 10 includes a device body 12, which defines an external surface 14. A surface layer 16 is attached to body external surface 14 and comprises an MCP-1 antagonist. As described more fully herein, surface layer 16 may be, for example, a unimolecular layer (e.g., a layer made entirely of MCP-1 antagonist molecules), or may be, for example, a layer that is one, or more, millimetres thick (e.g., a hydrogel layer that is one, or more, millimetres thick). Device body 12 and surface layer 16 may each include one or more therapeutic compositions that are released into a living body when device 10 is implanted therein. In the embodiment shown in FIG. 1, surface layer 16 has been partially removed to show device body 12 beneath. Device body 12 is indicated by hatching. FIG. 2 shows a transverse cross-sectional view of device 10. In one embodiment of device 10, shown in FIG. 3, surface layer 16 comprises a porous matrix 18 within which are disposed molecules 20 of an MCP-1 antagonist. Thus, in operation, device 10 is implanted into a portion of a living body where MCP-1 antagonist molecules 20 are released over time and reduce the foreign body response mounted by the body against implanted device 10.

It will be understood that the following description of the elements of device 10 is applicable to the corresponding elements of any medical device of the invention. Device body 12 can be made from any suitable material. Representative examples of synthetic polymers useful for making the device body include: (poly)urethane, (poly)carbonate, (poly)ethylene, (poly)propylene, (poly)lactic acid, (poly)galactic acid, (poly)acrylamide, (poly)methyl methacrylate and (poly)styrene. Useful natural polymers include collagen, hyaluronic acid and elastin.

Surface layer 16 can cover the whole of device body 12, or one or more parts of device body 12, such as areas of device body 12 where it is desired to reduce the foreign body response. Surface layer 16 can be made, for example, from any suitable material that: (a) permits deposition therein, or attachment thereto, of an amount of an MCP-1 antagonist, sufficient to reduce the foreign body response against medical device 10; and (b) can be attached to device body 12 (before or after deposition within, or attachment to, surface layer 16 of an amount of an MCP-1 antagonist sufficient to reduce the foreign body response against medical device 10). Representative examples of materials useful for making surface layer 16 include porous matrices and hydrogels. Surface layer 16 may comprise a variety of active agents in addition to an MCP-1 antagonist, such as for example, antibiotics, hormones, growth factors and other factors that enhance biocompatibility of implanted medical device 10.

Representative porous matrices useful for making surface layer 16 are those prepared from tendon or dermal collagen, as may be obtained from a variety of commercial sources, (e.g., Sigma and Collagen Corporation), or collagen matrices prepared as described in U.S. Pat. Nos. 4,394,370 and 4,975,527. One collagenous material is termed UltraFiber™, and is obtainable from Norian Corp. (Mountain View, Calif.). Certain polymeric matrices may also be employed if desired, including acrylic ester polymers and lactic acid polymers, as disclosed, for example, in U.S. Pat. Nos. 4,526,909, and 4,563,489. Particular examples of useful polymers are those of orthoesters, anhydrides, propylene-cofumarates, or a polymer of one or more α-hydroxy carboxylic acid monomers, (e.g., α-hydroxy acetic acid (glycolic acid) and/or α-hydroxy propionic acid (lactic acid)). Other synthetic polymeric porous materials useful for making surface layer 16 include, for example, silicone, polyurethane, polysulfone, cellulose, polyethylene, polypropylene, polyamide, polyester, polytetrafluoroethylene, and combinations thereof.

In the present invention, an amount of an antagonist of MCP-1 sufficient to reduce the foreign body response against device 10 is dispersed throughout a substantial portion of surface layer 16. Representative MCP-1 antagonists that can be dispersed throughout surface layer 16 include: antisense MCP-1 nucleic acid molecules, (such as antisense mRNA, antisense DNA or antisense oligonucleotides), dsRNA molecules, vectors containing transgenes encoding MCP-1 antagonists (such as MCP-1 ribozymes, RNAi and vectors that express MCP-1 blocking peptides), and polypeptides such as MCP-1 antibodies and blocking peptides.

In another embodiment, surface layer 16 is a hydrogel. A hydrogel can be any material forming a jelly-like product when suspended in a solvent, typically water or polar solvents. Despite the high degree of hydration of hydrogels, an important characteristic of hydrogels is that they are structurally stable. Such a hydrogel surface layer 16 can be coated onto device body 12 either covalently or noncovalently. Hydrogels can be made of natural or synthetic materials. Examples of natural hydrogels include fibrin, collagen, elastin, gelatin, pectin, and the like. Examples of synthetic hydrogels include polyacrylamides or poloxamers.

A hydrogel matrix comprising an MCP-1 antagonist may be coated on at least one surface, or on all surfaces, of device body 12. Representative MCP-1 antagonists that can be dispersed throughout a hydrogel include: antisense MCP-1 nucleic acid molecules, dsRNA molecules, vectors containing transgenes encoding MCP-1 antagonists (such as ribozyme transgenes which target MCP-1 mRNA, and MCP-1 blocking peptides), and may be formulated for optimal release of MCP-1 antagonist polypeptides such as MCP-1 antibodies and blocking peptides. For example, MCP-1 antibodies may be contained in a hydrogel reservoir and released over time in a controlled fashion. Surface layer 16 made from a hydrogel may also be used, for example, to deliver liposomes containing nucleic acid sequences encoding MCP-1 antagonists, as well as naked DNA encoding MCP-1 antagonists. There are reports of successful incorporation of enzymes and living cells into hydrogel matrices (e.g., U.S. Pat. Nos. 4,004,979, 4,452,892, 4,647,536 and 5,648,252) as well as successful incorporation of virus particles into hydrogel as a vaccine (U.S. Pat. No. 5,529,777). Therefore, in accordance with the invention, hydrogels are also useful to deliver MCP-1 antagonists incorporated in a viral vector such as, for example, adenovirus, AAV, vaccinia virus and retroviral vectors.

In another embodiment, body external surface 14 of medical device 10 is covalently activated and proteins are attached to surface 14 of the device to form surface layer 16. Covalent activation is useful, for example, to attach MCP-1 antagonists, such as blocking peptides and MCP-1 specific antibodies, to body external surface 14. Linkage of a protein to surface 14 can be accomplished by any technique that does not destroy the biological activity of the linked protein, for example, by attaching one or both ends of the protein to surface 14. Attachment may also be made at one or more internal sites in the protein. Multiple attachments (both internal and at the ends of the protein) may also be used. Body external surface 14 of implantable medical device 10 can be modified to include functional groups (e.g., carboxyl, amide, amino, ether, hydroxyl, cyano, nitrido, sulfanamido, acetylinic, epoxide, silanic, anhydric, succinimic, azido) for protein immobilization thereto. Coupling chemistries include, but are not limited to, the formation of esters, ethers, amides, azido and sulfanamido derivatives, cyanate and other linkages to the functional groups available on MCP-1 antagonist proteins or fragments. MCP-1 antagonist polypeptides can also be attached non-covalently by the addition of an affinity tag sequence to the protein, such as GST (Smith, D. B., and Johnson, K. S., Gene 67:31 (1988)), polyhistidines (Hochuli, E., et al., J. Chromatog. 411:77 (1987)), or biotin. Such affinity tags may be used for the reversible attachment of the protein to body external surface 14.

By way of representative example, MCP-1 blocking peptides can be attached to device body external surface 14, to form layer 16, by any of the following pairs of reactive groups (one member of the pair being present on surface 14, and the other member of the pair being present on the blocking peptide(s): hydroxyl/carboxylic acid to yield an ester linkage; hydroxyl/anhydride to yield an ester linkage; hydroxyl/isocyanate to yield a urethane linkage.

Body external surface 14 can be treated with radio-frequency discharge plasma (RFGD) etching to generate reactive groups in order to allow deposition of MCP-1 antagonists(s) thereon (e.g., treatment with oxygen plasma to introduce oxygen-containing groups; treatment with propyl amino plasma to introduce amine groups). When an RFGD glow discharge plasma is created using an organic vapor, deposition of a polymeric overlayer occurs on the exposed surface. RFGD plasma deposited films offer several unique advantages. They are smooth, conformal, and uniform. Film thickness is easily controlled and ultrathin films (10-1000 Angstroms) are readily achieved, allowing for surface modification of a material without alteration to its bulk properties. Moreover, plasma films are highly-crosslinked and pin-hole free, and therefore chemically stable and mechanically durable. RFGD plasma deposition of organic thin films has been used in microelectronic fabrication, adhesion promotion, corrosion protection, permeation control, as well as biomaterials. (see, e.g., Ratner, U.S. Pat. No. 6,131,580).

One of ordinary skill in the art will appreciate that surface layer 16 can include other molecules in addition to molecules 20 of an MCP-1 antagonist. For example, surface layer 16 can include drugs, growth factors, hormones, antibiotics, and other factors that enhance the biocompatibility of implanted medical device 10. Surface layer 16 can be configured in such a way as to optimize the timing of the delivery of the MCP-1 antagonist in order to reduce the foreign body response. For example, typically, antisense MCP-1 molecules are not fixedly attached to surface layer 16 so that the antisense molecules are free to diffuse out of surface layer 16 and be taken up by the cells of the surrounding tissue. Typically, however, MCP-1 peptide antagonists are fixedly attached, such as by covalent linkage to body external surface 14 or within surface layer 16 to prevent movement of the protein away from the implantation site.

In another aspect, the present invention provides methods for making a biocompatible implantable medical device, said methods comprising the step of forming a surface layer on an implantable medical device, wherein said surface layer comprises at least one MCP-1 antagonist, to yield a biocompatible medical device. The surface layer can be made, for example, from any suitable material that permits deposition on, or attachment thereto of an MCP-1 antagonist. Surface layer materials useful in the practice of this aspect of the invention-include porous matrices and hydrogels which are more fully described elsewhere in this patent application. Such surface layers can be formed separately from the medical device and attached to the device after formation, or the surface layer can be formed directly on the medical device such that the device is at least partially or completely encapsulated by the surface layer.

In the practice of the method of the invention, the surface layer comprises at least one MCP-1 antagonist. Representative MCP-1 antagonists useful in this aspect of the invention are described herein and include antisense MCP-1 nucleic acid molecules, dsRNA molecules, vectors encoding MCP-1 antagonists (such as ribozyme transgenes which target MCP-1 mRNA, and MCP-1 blocking peptides), and polypeptides such as MCP-1 antibodies and blocking peptides.

An MCP-1 antagonist may be disposed within (or upon) a surface layer, or attached to the surface of the device to form a surface layer. Methods of attaching surface layers to a medical device are further described herein. The MCP-1 antagonist may be disposed into a surface layer prior to, contemporaneously with, or subsequent to the formation of the surface layer on the medical device. For example, an MCP-1 antagonist may be disposed within a porous matrix or a hydrogel at any number of points between, and including the point of manufacture and the point of implantation of the device into a portion of a living body. In one embodiment, a surface layer can be formed on a device and the device can be stored and transported prior to incorporation of the MCP-1 antagonist. The MCP-1 antagonist may be disposed within a surface layer for example, by contacting or immersing the surface layer in an aqueous solution comprising an MCP-1 antagonist. By way of nonlimiting example, a plasmid encoding an MCP-1 blocking peptide described as SEQ ID NO: 4 may be disposed within a collagen matrix to form a gene activated matrix which is then coated onto an implantable medical device and stored prior to implantation.

The following examples merely illustrate the best mode now contemplated for practicing the invention, but should not be construed to limit the invention. All literature citations herein are expressly incorporated by reference.

### EXAMPLE 1

This example describes the decrease in chronic inflammation found at the site of subcutaneous implantation of biomaterial in MCP-1 null mice as compared to a wild type controls.

Generation of MCP-1-null mice: These mice were generated as described by Lu et al., 1998, J. Exp. Medicine 187:601-608.

Implantation of devices: Mixed cellulose ester filters (Millipore, 0.45 μm pore diameter) were used for implantation. Filters were implanted subcutaneously in the dorsal region of 10 MCP-1 null and 10 wild-type mice, for a total of 20 filters per experiment. Four weeks after implantation, the filters were excised en bloc, and processed for histological and immunohistochemical analysis.

Measurement of Chronic Inflammation: At four weeks post implant, the implants were removed en-bloc in an effort to not disturb the filter/host tissue interface. Chemically fixed explants were processed, embedded in paraffin and sectioned. Sections were stained with haematoxylin and eosin (H&E) and the number of foreign body giant cells (cells with three or more nuclei) per high power visual field (400× magnification) was determined.

Results: Quantitative analysis of foreign body giant cell formation in the MCP-1 null and wild-type mice revealed a 75% reduction in the number of foreign body giant cells surrounding the filters in the MCP-1 null mice as compared to wild-type controls. In addition, the surface of the filters in MCP-1 null mice was unaltered, whereas in wild-type mice it was significantly damaged. To address the possibility that the decrease in the number of foreign body giant cells was due to a deficiency in recruitment of monocytes, immunohistological analysis of sections was performed with anti-F4/80 antibody that recognizes an epitope on the surface of monocytes and non-activated macrophages. The anti-F4/80 antibody revealed an abundance of monocytes and macrophages in both wild-type and MCP-1 null mice. However, when the sections were stained with an anti-Mac 3 antibody that specifically recognizes foreign body giant cells, FBGC immunoreactive for Mac3 were absent in the MCP-1 null mice. These observations suggest that the recruitment of monocytes at the implant site were not compromised in MCP-1 null mice, but the process of macrophage fusion leading to the formation of foreign body giant cells is inhibited in the absence of MCP-1.

### EXAMPLE 2

This example describes a decrease in chronic inflammation at the site of implantation and a dramatic reduction in encapsulation of biomaterial implanted into the peritoneal cavity in MCP-1 null mice in comparison to wild-type control mice.

Implantation of Devices: Mixed cellulose ester filters (Millipore, 0.45 um pore diameter) were used for implantation. Filters were implanted into the peritoneal cavity of 10 MCP-1 null and 10 wild-type mice, for a total of 20 filters per experiment. Four weeks after implantation, the filters were retrieved from the peritoneum.

Measurement of Chronic Inflammation: At four weeks post implant, the implants were removed from the peritoneum and sections were stained with F4/80 and Mac3 antibodies. Whereas numerous F4/80 positive and Mac3 positively stained cells were present surrounding the implanted filter in the wild-type sections, there were very few positively stained cells in the MCP-1 null sections.

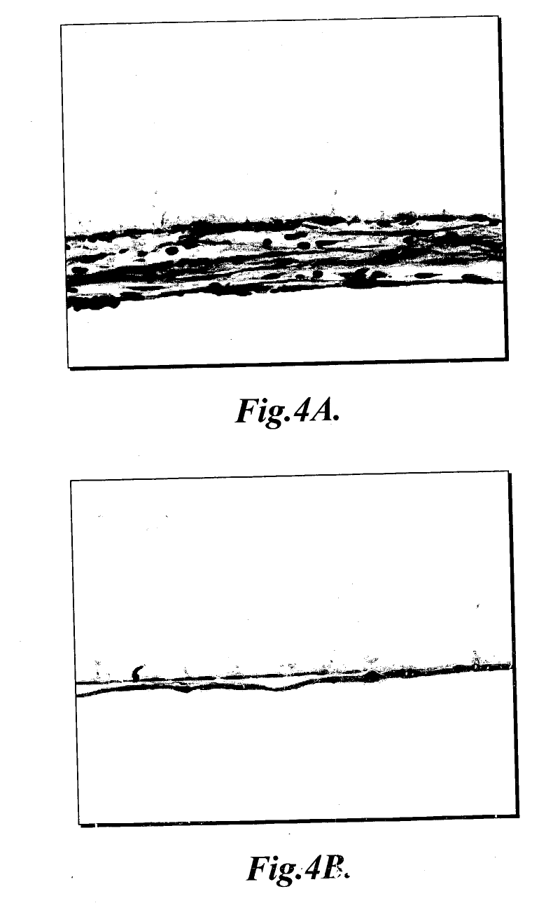

Measurement of Encapsulation: Representative sections of filters implanted intraperitoneally in wild-type and MCP-1 null mice were treated with Masson's trichrome to stain collagen fibers (collagen fibers stain blue and nuclei stain black) and are shown in FIGS. 4A and 4B (400× magnification). As shown in FIG. 4A, the representative implanted filter in the wild-type has significant encapsulation, whereas as shown in FIG. 4B, there is minimal encapsulation in MCP-1 null mice.

These results indicate that in the absence of MCP-1, implantation in the peritoneal cavity results in reduced chronic inflammation and reduced encapsulation of implanted biomaterial.

### EXAMPLE 3

This example describes the reduced biodegradation of subcutaneously implanted scaffolds observed in MCP-1 null mice as compared to wild-type mice.

Implantation of Devices: Alginate-based scaffolds were implanted subcutaneously into 10 wild-type and 10 MCP-1 null mice and retrieved after four weeks.

Histological Analysis: The retrieved scaffolds were sectioned and stained with Masson's trichrome to determine the extent of degradation and collagen deposition. Using this technique, collagen fibers stain blue and nuclei stain black. In the MCP-1 null mice, the scaffolds underwent minimal degradation with no collagen deposition or fibrovascular invasion and the majority of the cells present within the scaffold were mononuclear inflammatory cells. In contrast, in wild-type mice the scaffolds were invaded by a fibrovascular response, evidenced by formation of blood vessels, collagen deposition was observed, and numerous foreign body giant cells (multinucleated inflammatory cells) were observed.

These results indicate that scaffolds degraded at a slower rate in MCP-1 null mice, which could be attributed to the reduction in foreign body giant cells. Therefore, inhibition of expression or activity of MCP-1 may result in slower degradation of various biodegradable biomaterials such as scaffolds for tissue engineering applications.

### EXAMPLE 4

This example describes the decrease in chronic inflammation that occurs following subcutaneous implantation of a device coated with a gene activated matrix containing an plasmid expressing an MCP-1 antagonist as compared to a device coated with a control plasmid.

Methods and Materials: The plasmid pMT-7ND encodes a version of MCP-1 lacking amino acids 2-8, which is shown as SEQ ID NO: 4. The control plasmid used was the pCDNA 3 vector. The plasmid preparations were precipitated, washed and resuspended in endotoxin-free water prior to addition to the collagen solution.

Preparation of the Gene Activated Matrix (GAM): A 3.1 mg/ml collagen solution (Vitrogen 100, purchased from Cohesion) was neutralized with an equal volume of DMEM (GIBCO BRL) at 4° C. Plasmid DNA (pMT-7ND, pCDNA3) was added at a 1:1 (w/w) ratio and mixed with the collagen solution.

Preparation of Devices: 25-mm² Millipore filters (0.45-μm pore diameter, mixed cellulose ester) were soaked in 95% ethanol for 24 h, rinsed extensively with phosphate-buffered saline (PBS), and stored in endotoxin-free PBS. Filters were then immersed in 200 μl of GAM (500 μg collagen, 500 μg DNA) and incubated on a rotary shaker at 4° C. for 30 minutes. The preparations were then placed at −70° C. for 15 minutes and subsequently lyophilized.

Implantation of Devices: GAM coated filters were implanted subcutaneously in the dorsal region of wild type mice for a period of four weeks.

Analysis: After four weeks, filters were excised en bloc, fixed in zinc-buffered 10% formalin, and embedded in paraffin. Paraffin sections were stained with hematoxylin and eosin and the number of foreign body giant cells (FBGC) per unit length of filter was determined. The results, shown in FIG. 5 indicate that the filters coated with GAM expressing the truncated form of MCP-1 (pMT7-ND) exhibited a reduction in the number of foreign body giant cells per unit length of filter as compared to filters coated with a control vector. These results demonstrate that the truncated MCP-1 peptide can be used as an MCP-1 antagonist to increase the biocompatibility of a medical implant.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method of enhancing the biocompatibility of a medical device implanted within a portion of a living body, said method comprising contacting a portion of a living body that is in contact with an implanted medical device with an amount of an MCP-1 antagonist effective to inhibit a process selected from one of chronic inflammation induced by the presence of the medical device, and fibrous encapsulation of the medical device, thereby enhancing the biocompatibility of the medical device.

2. The method of claim 1 wherein said medical device is selected from the group consisting of wholly implanted medical devices and partially implanted medical devices.

3. The method of claim 1 wherein said MCP-1 antagonist and said device are separately introduced into the living body.

4. The method of claim 3 wherein said MCP-1 antagonist is introduced in a pharmaceutical composition.

5. The method of claim 1 wherein said medical device comprises a surface layer comprising said MCP-1 antagonist.

6. The method of claim 1 wherein said MCP-1 antagonist inhibits MCP-1 protein expression in tissues contacting the implanted medical device.

7. The method of claim 6, wherein said MCP-1 antagonist is selected from the group consisting of antisense MCP-1 nucleic acid molecules, an MCP-1 RNA inhibitor, double-stranded RNA molecules that cause RNA-mediated interference of MCP-1, and MCP-1 specific ribozymes.

8. The method of claim 7 wherein MCP-1 protein expression is inhibited by antisense MCP-1 nucleic acid molecules.

9. The method of claim 8, wherein the antisense MCP-1 nucleic acid molecules are at least seventy percent identical to the complement of an MCP-1 cDNA consisting of the nucleic acid sequence set forth in SEQ ID NO: 1.

10. The method of claim 8 wherein the antisense MCP-1 nucleic acid molecules are at least 100 bases in length and hybridize under stringent conditions to an MCP-1 cDNA molecule consisting of the nucleic acid sequence set forth in SEQ ID NO: 1.

11. The method of claim 8 wherein the antisense MCP-1 nucleic acid molecules are less than 100 bases in length and hybridize under stringent conditions to an MCP-1 DNA molecule.

12. The method of claim 7 wherein MCP-1 protein expression is inhibited by an MCP-1 RNA inhibitor.

13. The method of claim 7 wherein MCP-1 protein expression is inhibited by double-stranded RNA molecules that cause RNA-mediated interference of MCP-1.

14. The method of claim 7 wherein MCP-1 protein expression is inhibited by MCP-1 specific ribozymes.

15. The method of claim 1 wherein said MCP-1 antagonist inhibits MCP-1 protein activity in tissues contacting the implanted medical device.

16. The method of claim 15 wherein said MCP-1 antagonist is selected from the group consisting of an anti-MCP-1 antibody and an MCP-1 blocking peptide.

17. The method of claim 16 wherein an anti-MCP-1 antibody is introduced into the living body.

18. The method of claim 16 wherein a MCP-1 blocking peptide is introduced into the living body.

19. The method of claim 18 wherein the MCP-1 blocking peptide comprises the amino acid sequence set forth in SEQ ID NO. 4.

20. The method of claim 5, wherein said surface layer is one of a porous matrix and a hydrogel coating.

21. The method of claim 20, wherein said surface layer is a porous matrix.

22. The method of claim 20, wherein said surface layer is a hydrogel coating.

23. The method of claim 1, wherein said MCP-1 antagonist inhibits the process of chronic inflammation induced by the presence of the medical device.

24. The method of claim 1, wherein said MCP-1 antagonist inhibits the process of fibrous encapsulation of said implanted device.

25. An implantable medical device comprising:
(a) a device body; and
(b) a surface layer attached to the device body, said surface layer comprising an amount of an antagonist of MCP-1 sufficient to reduce a foreign body response against the device, wherein said device is adapted to be implanted within a portion of a living body.

26. The medical device of claim 25 wherein the device is selected from the group of devices consisting of wholly implanted medical devices and partially implanted medical devices.

27. The medical device of claim 25 wherein the surface layer attached to the device body comprises one of a porous matrix and a hydrogel coating.

28. The implantable device of claim 25, wherein said MCP-1 antagonist is selected from the group consisting of an antisense MCP-1 nucleic acid molecule, double-stranded RNA molecules that cause RNA-mediated interference of MCP-1, an anti-MCP-1 antibody, a MCP-1 blocking peptide and a MCP-1 ribozyme.

29. The implantable device of claim 28 wherein said MCP-1 antagonist comprises antisense MCP-1 nucleic acid molecules.

30. The implantable device of claim 29 wherein said antisense MCP-1 nucleic acid molecules are at least seventy percent identical to the complement of an MCP-1 cDNA consisting of the nucleic acid sequence set forth in SEQ ID NO: 1.

31. The implantable device of claim 29 wherein the antisense MCP-1 nucleic acid molecules are at least 100 bases in length and hybridize under stringent conditions to an MCP-1 cDNA molecule consisting of the nucleic acid sequence set forth in SEQ ID NO: 1.

32. The implantable device of claim 29 wherein the antisense MCP-1 nucleic acid molecules are less than 100 bases in length and hybridize under stringent conditions to an MCP-1 DNA molecule.

33. The implantable device of claim 28 wherein said MCP-1 antagonist comprises an MCP-1 RNA inhibitor.

34. The implantable device of claim 28 wherein said MCP-1 antagonist comprises double-stranded RNA molecules that cause RNA-mediated interference of MCP-1.

35. The implantable device of claim 28 wherein said MCP-1 antagonist comprises MCP-1 specific ribozymes.

36. The implantable device of claim 28 wherein said MCP-1 antagonist comprises an anti-MCP-1 antibody.

37. The implantable device of claim 28 wherein said MCP-1 antagonist comprises an MCP-1 blocking peptide.

38. The implantable device of claim 37 wherein the MCP-1 blocking peptide comprises the amino acid sequence set forth in SEQ ID NO. 4.

39. A method for making a biocompatible implantable medical device, said method comprising the step of making an implantable medical device comprising at least one external surface comprising a layer comprising at least one MCP-1 antagonist, to yield a biocompatible medical device.

40. The method of claim 39 wherein said MCP-1 antagonist is at least one of antisense MCP-1 nucleic acid molecules, double-stranded RNA molecules that cause RNA-mediated interference of MCP-1, anti-MCP-1 antibodies, MCP-1 blocking peptides and MCP-1 ribozymes.

41. The method of claim 39 wherein said layer comprises a porous matrix.

42. The method of claim 39 wherein said layer comprises a hydrogel.
